(19) United States
(12) Patent Application Publication
(10) Pub. No.: US 2024/0175015 A1
(43) Pub. Date: May 30, 2024

(54) RELIABLE IDENTIFICATION OF REGIONS ('A SITES') IN COMPLEX RNA MOLECULES THAT ARE ACCESSIBLE TO NUCLEIC ACIDS OR COMPLEXES OF NUCLEIC ACIDS WITH ENDONUCLEASES

(71) Applicant: Martin-Luther-Universität Halle-Wittenberg, Halle (Saale) (DE)

(72) Inventors: Prof. Dr. Sven-Erik BEHRENS, Halle (Saale) (DE); Torsten GURSINSKY, Halle (Saale) (DE); Selma GAGO-ZACHERT, Halle (Saale) (DE); Cornelia GRUBER, Berlin (DE); Vitantonio PANTALEO, Valenzano (BA) (IT); Ayson GHASEMZADEH, Halle (Saale) (DE)

(21) Appl. No.: 18/552,397

(22) PCT Filed: Mar. 23, 2022
(86) PCT No.: PCT/EP2022/057596

§ 371 Date: Sep. 25, 2023

(30) Foreign Application Priority Data
Mar. 25, 2021 (DE) 10 2021 107 508.4

(51) Int. Cl. C12N 15/11

(52) U.S. Cl. CPC C12N 15/111; C12N 2310/11; C12N 2310/141; C12N 2310/20; C12N 2320/11

(57) ABSTRACT

The invention relates to a method for detecting accessible regions (‘a sites’) in complex RNA molecules (target RNAs), wherein nucleic acids or complexes of these nucleic acids and endonucleases associated therewith bind to the a-sites and alter the function of the target RNAs where by nucleic acids of different types (‘eNAs’) bind to the a-sites and either alone or through an associated endonuclease alter the function of this target RNA. The invention further relates to the use of the method for identifying nucleic acids of different types eNAs that bind to the a-sites and are capable of directing endonucleases, selected from AGO proteins, RNase H and Cas proteins to the a-sites of target RNAs and are capable of, in the presence or absence of these endonucleases reliably affecting/altering the function of these RNA molecules; and to eNAs and a composition containing these eNAs in pathogen control.

### FIELD OF INVENTION

The invention relates to a method for detecting accessible regions (‘a-sites’) in complex RNA molecules (target RNAs), wherein nucleic acids or complexes of these nucleic acids and endonucleases associated therewith bind to the a-sites and alter the function of the target RNAs, characterized in that the method comprises the following steps:
(i) Provision of a target RNA:
(ii) esiRNA/ERNA screen that identifies siRNAs that can reliably induce a functional change in this target RNA in complexes with Argonaute (AGO) proteins:
(iii) subsequent assay with derived antisense DNA oligonucleotides (ASO) to determine whether they can induce a functional change in the target RNA in the presence or absence of RNase H and/or
(iv) subsequent testing with g/crRNAs derived therefrom to determine whether they can induce a functional change in the target RNA in the presence of a Cas protein: and
(v) Identification of a-sites in the target RNA, wherein nucleic acids of different types (‘eNAs’) bind to the a-sites and either alone or through an associated endonuclease alter the function of this target RNA.

The invention further relates to the use of the method for identifying eNAs capable of directing endonucleases selected from AGO proteins, RNase H and Cas proteins to the a-sites of target RNAs and, in the presence or absence of these endonucleases, preferably reliably affecting/altering the function of these RNA molecules: and eNAs and composition containing these eNAs in pathogen control.

### BACKGROUND OF THE INVENTION

Gene expression is regulated to a substantial extent at the level of complex ribonucleic acid molecules (RNAs), which are generated either in the course of transcription of the cellular genome (as pre-mRNAs or mRNAs) or in the course of viroid or viral replication (as viroids, viral genomes, viral mRNAs or viral antigenomes/replication intermediates). The RNAs in question, hereafter also referred to as ‘target RNAs’, are either self-replicating, as in the case of viroids, are replicated, as in the case of viral RNAs, are processed into mature RNAs, as in the case of pre-mRNAs and some viral RNAs, or are translated to generate proteins, as in the case of mature viral, prokaryotic or eukaryotic mRNAs and some viral genomes. However, target RNAs are also non-coding RNAs that modulate gene expression.

Various nucleic acid-based technologies have been developed to influence gene expression in order to modulate and/or inhibit it in a targeted manner. Different types of short-chain nucleic acids (NAs), also referred to as ‘directing NAs’ in the following, are associated with target RNAs via complementary base pairing and thus very specifically. The directing NAs can in turn be ribonucleic acid molecules such as small (small) sRNAs (e.g. small interfering RNAs, siRNAs, or microRNAs. miRNAs) or also guide (g) or CRISPR (cr) RNAs but also deoxyribonucleic acids such as antisense DNA oligonucleotides (ASO). The directing NAs can already inhibit the target RNA in its function, e.g. as a translation substrate, by binding (‘hybridization’) to it. In most cases, however, the directing NAs form a complex with an endonuclease. This endonuclease is either already associated with the directing NA and then becomes active after hybridization to the target RNA. Alternatively, after association of the directing NA to the target RNA, an endonuclease is recruited. The function of the target RNAs can then be inhibited via nuclease-mediated catalysis of an endonucleolytic cleavage. This is referred to as ‘silencing’ of the target RNA (1-6).

Problem statement. A core aspect of the application of directing NAs, among others for the purpose of directing endonucleases such as AGO proteins, RNase H or Cas proteins to a target RNA, is the accessibility of the target sequence to which the directing NAs are to hybridize. Thus, longer RNA molecules fold into complex secondary and tertiary structures where segments interact with adjacent or more distant segments. Thereby, ‘stems.’ ‘stem-loop,“kissing-loop’ and other RNA-RNA interactions form (7). Moreover, RNA-binding proteins associate with most RNA molecules in the cell (8). Accordingly, directing NA or NA/endonuclease complexes such as sRNA/AGO in RNA interference (RNAi), ASO/RNase H in antisense procedures, or g/crRNA/Cas in CRISPR/Cas procedures must compete with these structures and proteins to associate to the target (RNAi, antisense, and CRISPR/Cas procedures are discussed below). Accordingly, the more structured and inaccessible a target RNA is, the lower the activity of the NAs or nucleases. Conversely, the more accessible the target RNA, the more pronounced the effect. This relationship has been demonstrated for siRNA/AGO e.g. by Gago et al (9) and for ASO e.g. by Vickers and Crooke (10). It also became clear that on target RNAs comprising several kilobases, only very few regions (sites). which will be referred to below as a-sites (accessible sites), are well targetable for directing NA or nucleic acid/endonuclease complexes (see also previous work).

Previous attempts to reliably detect a-sites in complex RNA molecules (viral RNA genomes. viroids. cellular or viral mRNAs. non-coding RNAs, etc.) to achieve the highest possible silencing efficiency have been only moderately successful. For example. it is possible to determine the structure of an RNA in an aqueous environment or even in the cell by chemical modifications (11, 12). However. these approaches are very limited because they are technically complex and also provide RNA structures only under very defined conditions. If the conditions change-and this is constantly the case during the activity of an RNA in the cell. if only because of the dynamic interactions of RNA-binding proteins-experimental structure determination is not meaningful. For short regions. RNA structures can be predicted in silico using algorithms such as mfold. sfold. or RNAplfold. Similar approaches aim to detect regions accessible to directing NA or nucleic acid/endonuclease complexes. for example, via a ‘viral siRNA predictor’ (http://crdd.osdd.net/servers/virsirnapred). However. these programs. as well as approaches using libraries of directing NA. have limited reliability (13-20). Thus, the high patterning of long-chain target RNAs explains lack of silencing efficiencies in RNAi. ASO. and CRISPR/Cas approaches.

The identification of a-sites on target RNAs is thus of central importance for the use of methods that aim to regulate and/or inhibit the function(s) of complex RNA molecules. e.g. during gene expression or in the course of their replication via directing NAs or nucleic acid-directed endonucleases. If the accessibility of a target RNA can be determined precisely and reliably. i.e. via well-founded experimental methods. this allows on the one hand an efficient application of directing NAs such as sRNAs. ASO and g/crRNAs. On the other hand. it reduces the probability that the NAs or nucleic acid/endonuclease complexes in question are non-specifically active on other non-desired RNA molecules. These so-called ‘off-target effects’ have been a significant problem in the use of RNAi, antisense and CRISPR/Cas techniques to date: they occur. for example. via incomplete base pairing between directing NA and target RNA.

WO 2005042705 A2 describes a method for identifying. designing and synthesizing unique siRNA nucleotide sequences, which leads to the improvement of RNAi application. This involves comparing a database of mRNA sequences from the target species to an siRNA nucleotide sequence consisting of 18-25 nucleotides, where at least 1 1 consecutive nucleotides are complementary to the target mRNA sequence. In one embodiment. a miRNA sequence may be compared to a mRNA database.

WO 2019222036 A1 describes genetically modified proteins of the AGO family that achieve improved silencing. Special regulations with respect to the generation of the AGO proteins increase their efficiency. In one embodiment, the mutant AGO protein forms a complex with the guide strand of an siRNA, which leads to inhibition of gene expression. However, there is no mention of increased affinity. However, the present invention is not directed to the generation of mutant AGO proteins.

In WO 2019001602 A1, methods are provided for the identification and production of highly efficient siRNAs (esiRNAs ERNAs)in cytoplasmic extracts of Nicotiana tabacum. The system allows reconstitution of antiviral RNA silencing in vitro. Specifically, Dicer/DCL proteins and, optionally, AGO proteins are active in the described extracts. Thus provided esiRNAs/ERNAs find application in the regulation of gene expression in target organisms, including the enhancement of pathogen resistance.

### DESCRIPTION OF THE INVENTION

The object of the invention was to provide a method for detecting accessible regions (‘a-sites’) in complex RNA molecules (target RNAs), wherein directing nucleic acids or complexes of these nucleic acids and endonucleases associated therewith bind to the a-sites and reliably (‘reliably’) alter the function of the target RNAs.

The object of the invention is solved by a method according to claim 1.

In particular, the object of the invention is solved by a method for the detection of accessible regions, ‘a-sites’, in complex RNA molecules (target RNAs), wherein directing nucleic acids or complexes of these nucleic acids and endonucleases associated therewith bind to the a-sites and reliably alter the function of the target RNAs, characterized in that the method comprises the following steps:
(i) Provision of a target RNA:
(ii) esiRNA/ERNA screen, which identifies siRNAs that can reliably induce a functional change in this target RNA in complexes with AGO proteins:
(iii) subsequent assay with derived antisense DNA oligonucleotides (ASO) to determine whether they can induce a functional change in the target RNA in the presence or absence of RNase H and/or
(iv) subsequent assay with derived g/crRNAs to determine whether they can induce a functional change in the target RNA in the presence of a Cas protein.
(v) Identification of a-sites in the target RNA, wherein nucleic acids of different types (‘eNAs’) bind to the a-sites and reliably alter the function of the target RNA either alone or through an associated endonuclease.

### BRIEF DESCRIPTION OF THE FIGURES

FIG. 1A shows examples of consensus sequences of siRNAs determined to be optimal for plant AGO1 or AGO2.

FIG. 1B shows examples of ‘slicer assays’ performed with a 21 nt esiRNA and with consensus-optimized 21 nt esiRNAs.

FIG. 2 shows results of ‘slicer assays’ in BYL performed with an esiRNA and AGO/RISC complexes from human, plant (Nicotiana benthamiana), and fungus (Colletotrichum graminicola).

FIGS. 3A-3D show that a-sites accessible to sRNA/AGO/RISC are also accessible to ASO.

FIG. 4 shows the results of a ‘slicer assay’ performed with cytoplasmic HeLa S10 extract with a target RNA and matching siRNA or ASO in the presence and absence of translated human AGO2.

FIG. 5 shows the results of a CRISPR/Cas ‘cleavage’ experiment performed with BYL, Cas 13b and TBSV RNA compared to a ‘slicer assay’ with plant AGO protein.

### DETAILED DESCRIPTION

According to the invention, “target RNA” according to step (i) means complex ribonucleic acid molecules (RNAs) that are generated either in the course of transcription of a prokaryotic or eukaryotic cellular genome (as pre-mRNAs or mRNAs or non-coding RNAs) or in the course of viroid or viral replication (as viroids, viral genomes, viral mRNAs or viral antigenomes/replication intermediates). The RNAs in question are either self-replicating, as in the case of viroids, are replicated, as in the case of some viral RNAs, are processed into mature RNAs, as in the case of pre-mRNAs and some viral RNAs, or are translated to generate proteins, as in the case of mature viral, prokaryotic, or eukaryotic mRNAs and some viral genomes. However, target RNAs are also non-coding RNAs that modulate gene expression.

An esiRNA/ERNA screen according to step (ii), which identifies siRNAs that can reliably induce a functional change of a target RNA in complexes with AGO proteins and part of RNA-induced silencing complexes (RISC), is known in the prior art from WO 2019/001602. A functional change is understood to mean, for example, efficient endonucleolysis or inhibition of translation of the target RNA and thus, for example, silencing (loss of function) of this target RNA.

The esiRNA/ERNA screen according to step (ii) can be described as follows: A method established to date and described below aims at reliable RNAi-based functional modification of a target RNA. It finds application. for example. in the control of pathogens such as plant pathogenic viruses. The largest group of plant pathogenic viruses are (+)-stranded RNA viruses, whose genome consists of one or more positively oriented (mRNA sense) RNA molecules. Accordingly. after entry into the cell. the viral genome acts as messenger RNA (mRNA) from which viral proteins are translated. Some of these proteins. together with cellular host factors. then replicate the viral RNA. This produces double-stranded (ds) RNAs as replication products consisting of (+) and complementary (−)RNAs. From the (−)RNAs, in turn. mRNAs can be transcribed. from which further viral proteins are translated. The replication products. as well as double-stranded regions of the viral genome or viral mRNAs. can trigger an RNA interference mechanism (RNAi) of the infected plant host (see above). In the course of this RNAi immune response. a large number of small 19-25 nt long double-stranded siRNAs. a so-called ‘siRNA pool’. are generated by Dicer or Dicer-like proteins (Dicer/DCL) from the genome. from the replication products and from the mRNAs of the virus: in the maximum case. this pool consists of all siRNAs theoretically processable from these target RNAs by Dicer/DCL. Of these siRNAs, one strand. the ‘guide strand’, is bound by AGO. Guided by the guide strand. the siRNA/AGO complex then associates as part of a high-priority RISC to the ‘cognate’ viral RNAs. i.e. the target RNAs from which the siRNAs originally arose. The association of the siRNA/AGO/RISC alters the function of the target RNAs. e.g., by AGO-catalyzed endonucleolysis (hydrolysis) leading to inactivation (silencing) of the targets. However, as noted in several studies (9. 21-23). only a fraction of siRNAs from such a pool are active in the RNAi process on a viral target RNA. such as a viral genome or viral mRNA.

The reasons for this observation were addressed below: on the one hand. it was assumed that only a few of the siRNAs are efficiently incorporated into AGO/RISC. On the other hand. as explained. it was assumed that only a few a-sites on complex RNA molecules are accessible to RNAi. Then. in preliminary work. using the example of the (+)-stranded RNA virus. Tomato bushy stunt virus (TBSV). a multistep experimental method in vitro, the so-called ‘esiRNA/ERNA screen’, was established by which it became possible to identify such siRNAs, which are particularly efficiently antivirally active and will be called esiRNAs or ERNAs in the following. from a viral siRNA pool (Schuck J., Gursinsky T., Behrens S.- E. (2018). Method for targeted identification of highly efficient ‘small interfering RNAs. ERNAs’ for use in plants and other target organisms: WO 2019001602 A1).

The experimental basis for the esiRNA/ERNA screen is a cytoplasmic extract (BY2 lysate. BYL) prepared from protoplasts of suspension cells of the plant Nicotiana tabacum (24). BYL has endogenous Dicer/DCL activities, and it is possible to reconstitute SRNA/AGO/RISC complexes in vitro in this extract (25-28, 9). The esiRNA/ERNA screen was initially established for 21 and 22 nt siRNAs and the two plant AGO proteins AGO1 and AGO2: these forms of siRNAs and AGO protein variants are known to be involved in the plant antiviral RNAi immune response. Two properties served as indicators of an esiRNA/ERNA: efficient hydrolysis by AGO1 or AGO2 (‘slicing’ or ‘cleavage’) in vitro induced by this RNA on the target RNA of interest, and the ability to protect plants to which these siRNAs were administered in various ways (e.g. via ‘rub in’ or transient expression) against subsequent viral infection by silencing the viral RNA.

The esiRNA/ERNA Screen includes the following steps:
In the first step (‘DCL assay’), a target RNA is exposed to the Dicer/DCLs contained in the BYL. The siRNA pool generated from the target RNA by Dicer/DCL activity is detected in its entirety by next generation RNA sequencing (RNA-Seq).
In the second step (‘AGO-IP’), RISC are reconstituted using a selected AGO and a siRNA pool generated in BYL. The RISC are enriched via immunoprecipitation (IP) and the AGO-bound siRNAs are detected via RNA-Seq. Thus, the siRNAs of a pool that bind with high affinity to the respective AGO used are detected.
Finally, in the third step (‘slicer assay’), which can also be performed in medium-throughput format, the siRNAs identified in step two are tested individually with the respective AGOs used to determine whether they are able to induce efficient endonucleotic hydrolysis (‘slicing’ or ‘cleavage’) in the cognate target RNA.
In the final step, the esiRNAs identified in vitro will finally be tested in vivolin planta for their potential to protect plants against infection with the virus.

It has been shown that the esiRNA/ERNA screen makes it possible to identify esiRNAs from the siRNA pool of a viral RNA in vitro, i.e. in the ‘test tube’, via an empirical/experimental system, that are capable of effectively protecting plants against infection with the virus (9, WO 2019001602 A1).

In accordance with the invention, further findings were now obtained from which important open questions and new problems arose.
It was again clarified that the efficiency of an siRNA depends significantly on the binding affinity of the guide strand to the AGO protein. On the one hand, this affinity is determined by the 5′-nucleotide of the RNA: this fact was already known: For example, siRNAs with a 5′uridine bind preferentially to plant AGO1 and siRNAs with a 5′adenosine bind preferentially to plant AGO2 (29, 26, 9). Furthermore, according to the invention, it was found that the affinity of an sRNA to an AGO protein is determined by additional, previously unknown binding determinants in the sequence of the sRNA.
The efficiency of an siRNA also depends, as expected, on the accessibility of the target RNA (see above): Thus, accessible regions in TBSV RNA could be identified (26, 9). Interestingly, this did not yield clear correlations to an experimentally determined secondary structure of the TBSV genome (30): Thus, regions of the RNA that were assumed to be more accessible to RNAi according to the secondary structure were so de facto (i.e., in in vitro and in planta assays) only in very few cases. In contrast, other regions that should not be accessible according to the experimental structure were found to be cleavable by AGO1—or AGO2/RISC. This result confirmed the statement made in the problem statement that RNA structure determinations do not provide reliable information on a-sites of a target RNA. With the esiRNA/ERNA screen method, a method was thus established that empirically identifies siRNAs that hybridize, probably over their full length, to such a-sites and can direct the endonucleolytic or translation-inhibiting AGO/RISC there.
The efficiency of esiRNA-induced ‘cleavages’ of the respective target RNAs in the in vitro system correlates remarkably exactly with antiviral protection that can be achieved via application of these very esiRNAs in the plant (9, WO 2019001602 A1).

### Open Questions/Issues to be Resolved.

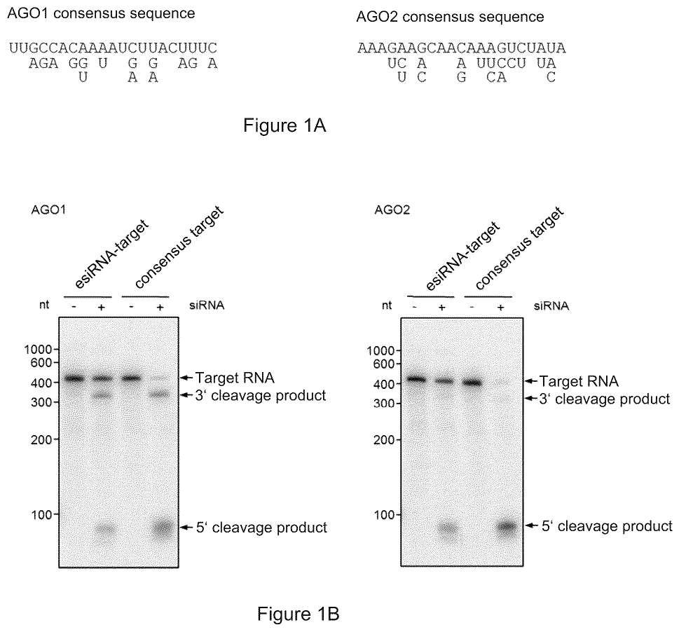

(a) The first open question/problem to be solved concerns the binding determinants of SRNAs, which, in addition to the 5′-nucleotide, determine the affinity of binding of a guide strand to an AGO protein. Since the affinity of an sRNA to AGO/RISC quite significantly also determines its activity (see above), knowledge of all binding determinants is of great importance for the identification of sRNAs that act efficiently and reliably in the RNAi process. Accordingly, the object was to fully define the binding determinants of an sRNA that are necessary for optimal association to AGO proteins.
(b) The second problem to be solved concerns the applicability of the esiRNA/ERNA screen procedure. To date, the procedure has been applied experimentally/empirically with 21 and 22 nt siRNAs and plant AGO1 and AGO2/RISC, as detailed. This identified esiRNAs/ERNAs that hybridize, either in plant AGO1/RISC or AGO2/RISC to different sites of a complex viral RNA (9). However, for broader application, it was important to move from empirical to rational use. In the foreground was the hypothesis that the a-sites contained in a complex folded target RNA may be accessible to any type of directing NA or nucleic acid/endonuclease complexes. Accordingly, the object was to extend and optimize the esiRNA/ERNA screen procedure in such a way that it is possible to identify a-sites in complex RNA molecules of any type that are universally accessible. i.e., accessible to all types of directing NA or nucleic acid/endonuclease complexes. Thus, even without knowing the exact structure of a complex RNAs, it should be possible to reliably identify the regions of these RNAs that can be used for functional modification or silencing of any kind.
   According to the Invention. These Objects were Solved as Follows:
(a) Improvements in the binding affinity of sRNAs to AGO. According to the invention, it could be shown that the binding affinity of an siRNA to an AGO protein is not solely determined by the type of 5′-nucleotide (29). as previously assumed. but significantly also by the remaining sequence of the siRNA. Thus, consensus sequences of guide strands of sRNAs that bind with high affinity to a specific AGO protein could be determined via a statistically relevant number of esiRNA/ERNA screen procedures: Thus. consensus sequences of siRNA guide strands that bind with high affinity to plant AGO proteins were determined. Similarly. consensus sequences of siRNAs that bind with high affinity to fungal AGO proteins were determined. Similarly, consensus sequences of siRNAs that bind with high affinity to mammalian AGO proteins were determined. By specifically adapting the sequence of a guide strand to the respective consensus sequence of the invention. the affinity of sRNAs for the AGO proteins concerned can thus be specifically increased and the activity of the AGO/RISC concerned in RNA-mediated silencing can be improved accordingly (FIG. 1B). Accordingly. the method according to the invention comprises an extended and improved step (ii). by which it is possible. via adaptation of the sequences of identified esiRNAs/ERNAs or other sRNAs (such as miRNAs) to the identified consensus sequences. to improve their activity in AGO/RISC. e.g. in RNA-mediated silencing procedures.
(b) Further development of the esiRNA screen method into a universally applicable ‘eNA-screen’ method. In the preliminary work it had become clear that the activities of esiRNAs/ERNAs on a target RNA in vitro and in vivo (i.e. in the specific case in the plant) correlate in very good agreement (see above). Accordingly. it was deduced from this observation that the environment in the cytoplasmic extracts used in vitro must be such that complex RNA molecules, including the RNA-binding proteins present there. fold in a very similar manner as in the intact cell or organism. This notion is supported by previous studies in which it had been shown that viral RNA replication can also be reconstituted in BYL. Similar to the RNAi process, viral replication can only occur when the folding of viral RNA can form optimally (24.31).

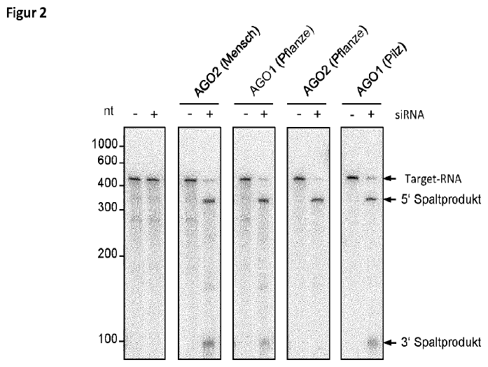

The postulate was subsequently confirmed, and based on this, the esiRNA/ERNA screen method was further developed in terms of the invention such that it is now possible to identify a-sites in complex RNA molecules that are accessible to different directing NA or nucleic acid/endonuclease complexes (FIGS. 2-5).

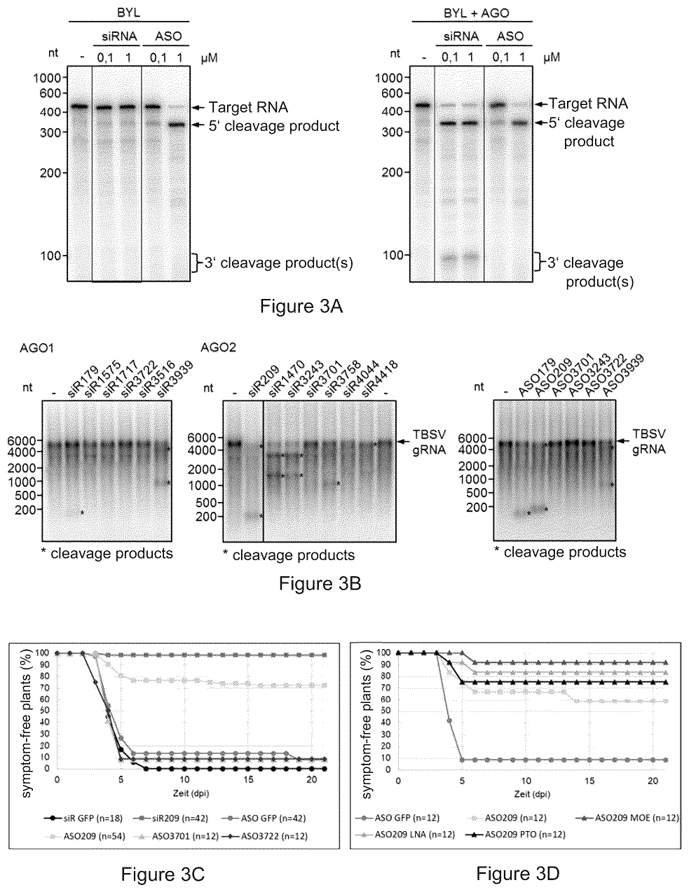

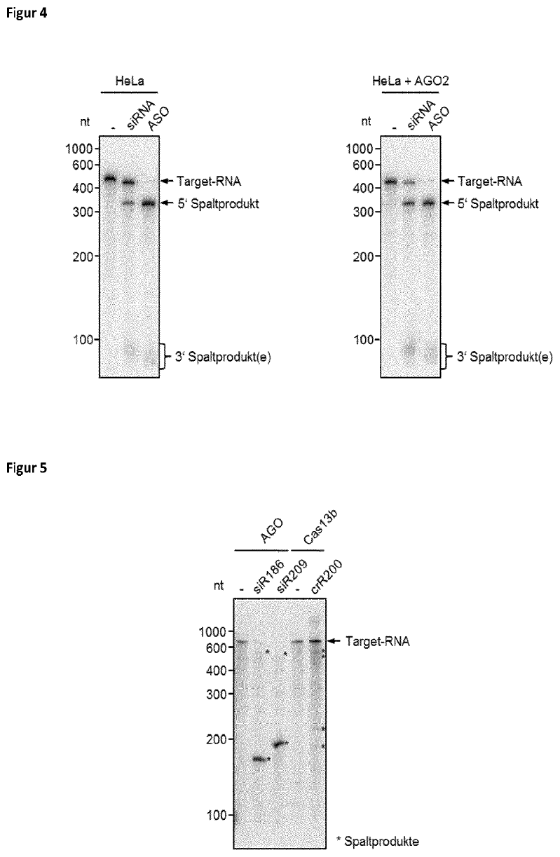

In Accordance with the Invention, the Following was Found:
If the sequence of the guide strand of an sRNA is adapted to the respective binding preferences of AGO proteins of different species via the 5′-nucleotide and/or via the consensus sequence (see above), the resulting sRNA/AGO/RISC act exactly identically on a complex target RNA. This was demonstrated in in vitro slicer assays. Here, AGO/RISC were reconstituted with AGO proteins from species of three different kingdoms of the eukarya domain, namely AGO from Plantae, AGO from Fungi. and AGO from Mammalia in BYL. The different AGO/RISC were thereby reconstituted with the same, respectively adapted sRNA (see above). This sRNA was an esiRNA/ERNA that can bind to an a-site of a target RNA. When tested on this same target RNA, identical slicing cleavage (hydrolysis) of the target RNA was observed with all AGO/RISCs (FIGS. 3A-3D). This unexpected finding implies that sRNAs that induce slicing cleavage or inhibition of translation on a target RNA associated with a particular AGO/RISC (e.g., from plant) do so in the same way on the same target RNA in another AGO/RISC (e.g., from fungus) derived from a completely different species (FIGS. 3A-3D). This demonstrated that a-sites of complex RNA molecules are universally accessible to AGO/RISC of any nature, provided that these AGO/RISC have bound an sRNA that can hybridize to this a-site.
The findings described above were obtained with both the plant cytoplasmic BYL extracts and cytoplasmic extracts from human cells (HeLa S10).
According to the invention, this was shown for target RNAs of different nature, e.g. genomic RNA from viruses as well as mRNAs of different organisms originating from viruses, bacteria, plants, fungi, oomycetes, animals such as nematodes, arthropods and higher animals such as mammals, or humans.

Thus, it was shown in accordance with the invention that sRNA/AGO/RISC of different species, when incorporated with identical or very similar binding sequence on the same target RNA, act identically and that this is equally the case in cytoplasmic extracts from plants and humans. Thus, it could be concluded that complex RNA molecules apparently fold similarly in the cytoplasmic extracts of different cell types and that a-sites of these RNA molecules are accordingly formed equally in each case. Thus, an a-site is equally accessible to AGO/RISC of different origin, provided that they have incorporated an sRNA that can hybridize to this a-site.

From these findings, it was further postulated that it should be analogously possible to direct other endonucleases such as RNase H or Cas to the a-sites of complex RNA molecules, which apparently form very reproducibly under the in vitro conditions. The a-sites should thus be universally identifiable and usable, i.e. not only for RNAi, but also for DNA-mediated and CRISPR/Cas-mediated silencing.
This could be demonstrated according to the invention: By using ASO or g/crRNAs that were sequence aligned to previously identified esiRNAs/ERNAs, either RNase H or Cas could indeed be directed to and become active on a corresponding target RNA (FIGS. 3A-3D and 4).
The findings described above were again obtained with both the plant cytoplasmic BYL extracts and cytoplasmic extracts from human cells (HeLa S10) (FIG. 5).

Regions, a-sites, of a target RNA that are accessible to sRNA/AGO/RISC are correspondingly also accessible to ASO/RNase H as well as to g/crRNA/Cas. According to the invention, a basic principle is thus disclosed which shows that directing NAs such as sRNAs, ASO and g/crRNAs or the nucleic acid/protein complexes derived therefrom SRNA/AGO/RISC, ASO/RNase H and g/crRNA/Cas associate according to analogous principles to accessible a-sites of a complex target RNA and become reliably effective there.
According to the invention, the esiRNA screen method can thus be extended and used as an ‘eNA-screen’ method (eNA stands for effective directing nucleic acid). For example, screening with sRNA/AGO/RISC can now be used to identify regions in a complex RNA on which ASO/RNase H and g/crRNA/Cas are also active when similar or identical sequences of ASO or g/crRNAs are used (see application examples). This is particularly evident when using viral target RNAs: for example, viral replication in plants can be inhibited by siRNAs that bind to a-sites of the viral RNA as well as by ASO or g/crRNAs of the same or similar sequence (see FIGS. 3A-3D and 4 and Working Examples).
Accordingly, the method of the invention comprises, as step (iii), a subsequent assay with antisense DNA oligonucleotides (ASO) derived from the esiRNAs/ERNAS identified in step (ii) to determine whether they can induce a functional change in the target RNA in the presence or in the absence of RNase H.
In addition, the method according to the invention comprises as step (iv) an assay, subsequent to steps (ii) or (iii), with g/crRNAs derived from the esiRNAs/ERNAs or ASO identified in steps (ii) or (iii) for whether they can induce a functional change of the target RNA in the presence of a Cas protein.
Thus, a-sites are identified in this target RNA to which directing nucleic acids of different types (‘eNAs’) bind and can reliably alter the function (e.g. see above) of this target RNA either alone or through an associated endonuclease.

Based on the aforementioned observations, experimental systems are thus provided according to the invention with which it is reliably possible to detect regions, a-sites, in complex target RNAs that are accessible to endonuclease-directing nucleic acids such as siRNAs, miRNAs, ASO, and g/crRNA. The information obtained can be used to exploit RNA-mediated. DNA-mediated and CRISPR/Cas-mediated silencing more efficiently and specifically than previously possible (i.e. with significantly reduced risk of ‘off targeting’) for pathogen control and/or modulation of gene expression processes in cells.
Accordingly, the method of the invention includes as step (v) the identification of a-sites in the target RNA, wherein nucleic acids of different types (‘eNAs’) bind to the a-sites and reliably alter the function of this target RNA either alone or through an associated endonuclease.

As mentioned above, three major technologies are available in which nucleic acids, i.e., ribonucleic acids (RNAs) or deoxyribonucleic acids (DNAs) are used as effectors, i.e., directing NAs, for functional changes of complex target RNAs such as silencing:
(i) RNA-mediated silencing (also referred to as RNA interference, RNAi), mediated, among others, by microRNAs (miRNAs) or small interfering RNAs (siRNAs), which, associated with Argonaute (AGO) endonucleases, are active in RNA-induced silencing complexes (RISC).
(ii) DNA-mediated silencing mediated by various forms of antisense DNA oligonucleotides (ASO), which, among other things, can activate the RNA degrading enzyme RNase H.
(iii) Clustered regularly interspaced short palindromic repeats associated (Cas)-mediated regulation (CRISPR/Cas) mediated via guide/CRISPR RNAs (g/crRNAs) associated to a Cas endonuclease.

The three nucleases mentioned, AGO, RNase H, and Cas, presumably originate from an RNase H-like precursor and act in a similar endonucleolytic manner (6).

RNA-mediated silencing, RNAi. RNAi is a cellular mechanism that post-transcriptionally regulates gene expression in eukaryotes (32) In plants, as well as fungi, oomycetes, nematodes, and insects, RNAi is also a central component of immune defense (33, 34). RNAi is regulated by partially or completely double-stranded small non-coding RNAs (sRNAs), which are usually between 20 and 30 nucleotides (nt) in length. Central regulators at the post-transcriptional level are microRNAs (miRNAs) and small interfering RNAs (siRNAs). miRNAs are genomically encoded, transcribed in the form of precursor molecules, and maturated by ribonucleases, including Dicer/DCL. siRNAs are processed from double-stranded (ds) RNA elements by Dicer/DCL proteins. These dsRNA elements can be structured regions from genomes or replication products of viroids or RNA viruses, but can also originate from mRNA molecules and other forms of RNA molecules, such as non-coding RNAs. Active miRNAs and siRNAs become active in RISC, whose main component is AGO proteins. In this process, the guide strand of the sRNA is bound by AGO, while the other strand of the sRNA double strand is removed/degraded. The guide strand in the sRNA/AGO/RISC complex hybridizes to regions of the target RNA. In the case of miRNAs, these are, for example, partially or fully complementary regions on target mRNAs: in the case of siRNAs, these are fully complementary regions on all types of target RNAs, such as genomic RNAs or mRNAs from pathogens from which these siRNAs were originally generated (so-called ‘cognate RNAs’). The target RNAs can then be inactivated, e.g. by inhibition of translation or by endonucleolytic hydrolysis (slicing cleavage) catalyzed by AGO, sometimes with subsequent degradation of the cleavage (cleavage) products by exonucleases. Thus, the activity of RISC-associated sRNAs may result in target-specific inhibition (silencing) of gene expression (34-40).

RNAi has been used for experimental regulation of gene expression at the (pre)mRNA level and to combat pathogens for about two decades. For this purpose. 21 or 22 bp long siRNAs are predominantly used (41): these consist of a completely complementary RNA duplex with two base overhangs at each of the 3′ ends (35, 36). Thus, there are both transgenic and nontransgenic (‘transient’) RNAi approaches in plants that target viral infections. RNAi is also used to control plant pathogenic insects, nematodes, oomycetes, fungi or plant pathogenic plants. Here, for example, mRNAs of essential proteins of these pathogens are used as targets and the pathogens' RNAi mechanism is directed against their own mRNAs. In virtually all eukaryotic organisms, including mammals, RNAi-based approaches can be used to target and regulate cellular gene expression at the RNA level. However, specifically in mammals, they are also being used to target pathogens, including animal and human pathogenic viruses, some of which are already in clinical trials (40-43). In 2018, the first siRNA-based drug. ‘patisiran’, was approved in humans for use against polyneuropathy in hATTR (hereditary transthyretin-mediated amyloidosis).

In the course of the past years. the technology has been improved considerably. Chemical modifications of the siRNAs have been and continue to be decisive driving forces: for example. 2′-ribose modifications (2′-O-methyl. 2′-fluoro. 2′-O-(methoxyethyl) (2′-MOE)) have led to Decreases in immunogenicity. increases in resistance to endo- and exonucleases. and increases in the melting temperature of the siRNA/target RNA hybrid can be achieved. Other sugar modifications (‘locked’ (LNA) or unlocked. (UNA)) increase or decrease the binding affinity of siRNA to the target RNA. ‘Backbone’ modifications such as phosphorothioates (PS) or ‘peptide nucleic acids’ as well as sugar phosphate modifications such as morpholino/PMO (phosphorodiamidate morpholino) affect hydrophobicity. protein association and again nuclease resistance or hybridization temperature. Because delivery of siRNAs into cells or target organs is still difficult for many applications. modifications and formulations also aim to deliver the negatively charged NAs specifically to the target cells and. after endocytosis. through the membrane to the site of action in the cytoplasm (42. 44).

DNA-mediated silencing. This includes methods that artificially and specifically influence the metabolism and/or expression of target RNAs via the use of single-stranded. 12-30 nucleotide long. chemically synthesized antisense DNA oligonucleotides (ASO). Unlike miRNAs or siRNAs. which post-transcriptionally regulate gene expression in cellular processes. chemically synthesized 12-30 nt long single-stranded DNA oligonucleotides have been used from the beginning for the purpose of targeted silencing of RNA molecules. Hybridization of an ASO to the complementary sequence of a target RNA can influence its function in different ways: In the steric blocking mechanism. the binding of the ASO to the target RNA alone inhibits its translation, the binding of a regulatory protein and/or its replication. In the degradation pathway. the binding of ASO to the target RNA leads to the recruitment of the enzyme RNase H. which then hydrolyzes the target endonucleolytically at the RNA/DNA hybrid. Thus. for example. binding of ASO to mRNAs can inhibit their translation, and binding to viral genomes can inhibit their translation and/or replication (45).

In experimental and therapeutic indications. applications of ASO are predominantly aimed at degrading target RNAs through the degradation mechanism (45). The active enzyme in this regard is RNase H. which is present in mammalian cells in the nucleus and. to a lesser extent. in the cytoplasm. The enzyme is involved in cellular DNA replication and repair: for example. RNase H recognizes the structure of at least five bp-encompassing DNA/RNA hybrids in a sequence-independent manner (46). On these. it presumably acts similarly to a DNase (6). The 5′-phosphate/3′-hydroxyl products formed during cleavage are degraded in the cell by exonucleases. the rate of degradation again depending on the chemistry of the ASO (see below), but also on the type of target RNA attacked (47). ASO that are to utilize the degradation mechanism must accordingly contain at least five consecutive nt that hybridize to the target. with longer complementary sequences significantly increasing specificity.

As with siRNAs. the properties of ASO can also be significantly influenced via chemical modifications. This concerns. among other things. pharmacokinetics. stability and toxic/promoting inflammatory properties. In addition. uptake into defined target cells. binding affinity to target RNA. and binding affinity to proteins can be modulated (45. 48. 49). Thus. conjugates such as GalNac3 or cholesterol as well as sugar modifications such as 2′-MOE are in use. In addition, bridged nucleic acids such as LNA or cEt BNA are used. Finally. base and backbone modifications with similar effects as described above for siRNAs are also used. The type of chemical modification also determines whether an ASO operates via steric blocking or degradation. PMOs. for example, which have been tested in a number of antiviral approaches (50). cause blockage of the target RNA exclusively.

So-called ASO gapmers have proven to be particularly effective in therapeutic approaches in humans, where an RNase H-active region at each 5′- and 3′-end is flanked by nuclease-resistant. chemically modified domains such as LNA or 2′-O-modified nucleotides (48). Chemically modified PS-ASOs can pass through the membrane into the cytoplasm of a target cell after endocytosis ‘gymnotically’. i.e., even without an additional carrier. via the hydrophobicity of the phosphorothioate ‘backbone’. Since 2016. PS/MOE-modified ASO have been used as ‘nusinersen’ in a first ASO-based therapy against spinal muscular atrophy. In this context, nusinersen inhibits an aberrant splicing process (48).

CRISPR/Cas-mediated silencing. The CRISPR/Cas system is a central component of the adaptive immune system of bacteria and archaea. particularly directed against phage infections and plasmid DNA uptake. Thus. catalyzed by various Cas proteins. portions of genomic phage DNA or plasmids are integrated into the bacterial genome as so-called protospacers between direct repeats. i.e. identical. repetitive sequences. From this DNA segment. pre-CRISPR RNAs (pre-crRNAs) are transcribed and these are processed by further Cas proteins into CRISPR RNAs (crRNAs): in different microbial species, three very different mechanisms of processing exist here. Mature crRNAs then associate in a third step either directly with the processing or other Cas proteins and form interfering complexes that can then. depending on the sequence of the crRNAs. associate with complementary DNA or RNA elements and hydrolyze them endonucleolytically. Via specific constructions of crRNAs in the form of so-called guide RNAs (g/crRNAs). Cas proteins can be targeted to DNA or RNA molecules, e.g. for the purpose of endonucleolytic hydrolysis, in order to specifically influence gene expression at the transcriptional as well as at the post-transcriptional level (51). For example, Cas13a from Leptotrichia shahii (LshCas13a) or Leptotrichia wadei (LwaCas13a) have been elicited for silencing RNA molecules. Similar to siRNAs and ASO, g/crRNAs are also used in a chemically modified manner to modulate the binding properties and also the stability of the RNA molecules (52-54).

It is a particular advantage of the invention that it is now possible to identify a-sites in different complex target RNAs, whereby nucleic acids of different types (‘eNAs’) bind to these a-sites and either alone or through an associated endonuclease reliably alter the function of this target RNA. In a particularly preferred embodiment of the method according to the invention, a-sites are therefore identified in the target RNA, whereby nucleic acids of different types (“eNAs”) bind to these a-sites and either alone or through an associated endonuclease reliably change the function of this target RNA. Reliable′ in the sense of this particularly preferred embodiment of the method of the invention means that the eNAs at the identified a-sites always bring about a corresponding change in function of the target RNA.

The eNAs of different types are preferably selected from siRNAs (e.g. siRNAs, vsiRNAs, tasiRNAs, hpsiRNAs, natsiRNAs, vasiRNAs, hetsiRNAs, piwi RNAs, easiRNAs, phasiRNAs, endo-siRNAs) miRNAs, antisense DNA oligonucleotides, and g/crRNAs, preferably any type of said eNAs. such as. e.g. naturally occurring, synthetic, recombinant or artificially produced eNAs.

In another preferred embodiment of the invention, the endonucleases are selected from AGO, RNase H and/or Cas proteins. In connection therewith, it is further preferred that the, particularly preferably reliable, modification of the function of the target RNA is mediated by RNA, DNA or CRISPR/Cas.

As mentioned above, functional change of the target RNA is understood to mean, for example, efficient endonucleolysis or inhibition of translation of the target RNA and thus silencing (loss of function) of this target RNA. Preferred according to the invention is the (reliable) silencing of the target RNA. In a particularly preferred embodiment of the method according to the invention, (reliable) silencing of the target RNA is understood, wherein the silencing is selected from RNA, DNA or CRISPR/Cas-mediated silencing.

It is particularly preferred if the eNAs (sRNAs, antisense DNA oligonucleotides (ASO) or g/crRNAs) identified in steps (ii), (iii) and/or (iv) of the method according to the invention have a consensus sequence. This has the advantage that the corresponding endonuclease works more effectively, e.g. by the consensus sequence increasing the affinity of the eNA to this endonuclease.

In a particularly preferred embodiment of the invention, the eNA is an siRNA. Examples of suitable consensus sequences of the guide strand of the siRNA provided according to the invention are selected, e.g., from.
i) UUX₁X₂X₃AX₄X₅AX₆AUX-UX₈ACX₉X₁₀UX₁₁ (SEQ ID No. 1)
wherein
X₁ is selected from G and A;
X₂ is selected from C and G;
X₃ is selected from C and A;
X₄ is selected from C and G;
X₅ is selected from A, G and U;
X₆ is selected from A and U;
X₇ is selected from C, G and A;
X₈ is selected from U, G and A;
X₉ is selected from U and A;
X₁₀ is selected from U and G; and
X₁₁ is selected from C and A;

and
ii) AAAX₁₂X₁₃AX₁₄CAAX₁SAX₁₆X₁₇X₁₈X₁₉X₂₀UX₂₁X₂₂A (SEQ ID No.: 2)
wherein
X₁₂ is selected from G and U;
X₁₃ is selected from A, C and U;
X₁₄ is selected from G, A and C;
X₁₅ is selected from C, A and G;
X₁₆ is selected from A and U;
X₁₇ is selected from A, U and C;
X₁₈ is selected from G, C and A;
X₁₉ is selected from U and C;
X₂₀ is selected from C and U;
X₂₁ is selected from A and U; and
X₂₂ is selected from U, A and C;

Preferred consensus sequences are the sequences UUGCCACAAAAUCUUACUUUC (SEQ ID No: 3) and AAAGAAGCAACAAAGUCUAUA (SEQ ID No: 4).

In a very particularly preferred embodiment of the method according to the invention. the eNA is an sRNA. i.e. any form of siRNAs (e.g. siRNAs. vsiRNAs. tasiRNAs. hpsiRNAs. natsiRNAs. vasiRNAs. hetsiRNAs, piwi RNAs. easiRNAs, phasiRNAs. endo-siRNAs) or any form of miRNAs and the endonuclease is an AGO protein. Argonaute proteins (AGO proteins) are a family of proteins whose representatives are evolutionarily highly conserved. They are found in almost all organisms. where they play an important role in the activation and regulation of gene expression. AGO family representatives are involved in transcriptional gene silencing (TGS) and posttranscriptional gene silencing (PTGS) (37). The eNAs are particularly suitable for RNA-mediated silencing of target RNAs. thus for silencing RNAs that may be of cellular but also pathogenic origin.

In another very particularly preferred embodiment of the method according to the invention. the eNA is any form of antisense DNA oligonucleotide (ASO) and the endonuclease is an RNase H. Ribonucleases H (from ribonuclease hybrid. synonymously RNase H) comprise a group of enzymes (ribonucleases) that degrade RNA into DNA-RNA hybrids. They are divided into two groups. RNase HI (in bacteria) or RNase H1 (in eukaryotes) and RNase HII (in bacteria) or RNase H2 (in eukaryotes). Type H ribonucleases are found in almost all living organisms and are sequence-nonspecific endonucleases that hydrolyze the phosphodiester bond of RNA in double strands of DNA and RNA. resulting in a 3′-hydroxy group and a 5′-phosphate group (6). These eNAs are particularly suitable for DNA-mediated silencing of target RNAs. thus for silencing RNAs that may be of cellular but also pathogenic origin.

In another particularly preferred embodiment of the method according to the invention, the nucleic acid is any form of CRISPR RNA or a CRISPR RNA-derived g/crRNA and the endonuclease is a Cas protein. The DNA- or RNA-cleaving enzyme called Cas (from English CRISPR-associated ‘CRISPR-associated’) binds a specific RNA sequence. Before or after this RNA sequence is another RNA sequence that can bind by base pairing to a target DNA or target RNA with complementary sequence. The g/crRNA serves as a bridge between Cas and the target DNA or target RNA to be cleaved. The concatenation of the Cas enzyme. the g/crRNA. and the target DNA or target RNA brings the Cas endonuclease into close spatial proximity to the target. whereupon Cas hydrolyzes it (51). These eNAs are particularly suitable for Cas-mediated silencing of target RNAs. thus for silencing RNAs that may be of cellular but also pathogenic origin.

Examples of suitable eNAs provided by the invention are selected from the sequences shown in Tables 1. 2. and 3.

TABLE 1
Examples of siRNAs suitable as eNAs.

| | Sequence guide | SEQ | Sequence passenger | SEQ |
|---|---|---|---|---|
| siRNA | strand (5′-3′) | ID. | strand (5′-3′) | ID. |
| siR | UAGUUCAUCCAUGCCAUG | 5 | ACAUGGCAUGGAUGAACU | 6 |
| GFP | UGU | | AUA | |
| siR179 | UGAUGGUCUCCAUGUCGC | 7 | AGCGACAUGGAGACCAUC | 8 |
| | UUG | | AAG | |
| siR186 | AUUCUCUUGAUGGUCUCC | 9 | UGGAGACCAUCAAGAGAA | 10 |
| | AUG | | UGA | |
| siR209 | AAAUCUCUUUCUUAGGCC | 11 | UGGCCUAAGAAAGAGAUU | 12 |
| | AAA | | UUU | |
| siR1470 | AUAUGCAGACUCUCCACG | 13 | CCGUGGAGAGUCUGCAUA | 14 |
| | GCU | | UCA | |
| siR1575 | UUUCGAGGCUGAAUCACC | 15 | GGGUGAUUCAGCCUCGAA | 16 |
| | CGA | | ACC | |
| siR1717 | UUUAGCCCGGAAAAUUGC | 17 | UGCAAUUUUCCGGGCUAA | 18 |
| | ACC | | AUG | |
| siR3243 | AUUCGCCAACUCAACUCU | 19 | UAGAGUUGAGUUGGCGAA | 20 |
| | AUC | | UUA | |
| siR3516 | AGAUGCUGUGACAAGAGC | 21 | CGCUCUUGUCACAGCAUC | 22 |
| | GCC | | UGG | |
| siR3701 | AAAAACGCACUGUCUGUA | 23 | GUACAGACAGUGCGUUUU | 24 |
| | CCU | | UCA | |
| siR3722 | UUAGAGACAGUACAAUUU | 25 | UAAAUUGUACUGUCUCUA | 26 |
| | AUG | | ACC | |
| siR3758 | AUACCGGUAGAUGUGAAU | 27 | CAUUCACAUCUACCGGUA | 28 |
| | GUC | | UCA | |
| siR3939 | UUCACUGUUAGCUUGUUC | 29 | GGAACAAGCUAACAGUGA | 30 |
| | CCU | | ACG | |
| siR4044 | AUUCGAAUUCGUCUCAUC | 31 | CGAUGAGACGAAUUCGAA | 32 |
| | GUU | | UCA | |
| siR4418 | AAGAGUCUGUCUUACUCG | 33 | GCGAGUAAGACAGACUCU | 34 |
| | CCU | | UCA | |

TABLE 2
Examples of antisense DNA oligonucleotides (ASO)
suitable as eNAs.

| ASO | Sequence (5′-3′) | SEQ ID |
|---|---|---|
| ASO GFP | TAGTTCATCCATGCCATG | 35 |
| | TGT | |
| ASO179 | TGATGGTCTCCATGTCGC | 36 |
| | TTG | |
| ASO209 | AAATCTCTTTCTTAGGCC | 37 |
| | AAA | |
| ASO3243 | ATTCGCCAACTCAACTCT | 38 |
| | ATC | |
| ASO3701 | AAAAACGCACTGTCTGT | 39 |
| | ACCT | |
| ASO3722 | TTAGAGACAGTACAATT | 40 |
| | TATG | |
| ASO3939 | TTCACTGTTAGCTTGTTC | 41 |
| | CCT | |
| ASO209 | [2′-O-MOE-rA][2′-O-MOE-rA] | 42 |
| MOE | ATCTCTTTCTTAGGCCA | |
| | [2′-O-MOE-rA][2′-O-MOE-rA] | |
| ASO209 | [LNA-A] | 43 |
| LNA | AATCTCTTTCTTAGGCCAA | |
| | [LNA-A] | |
| ASO209 | A*A*A*T*C*T*C*T*T*T* | 44 |
| PTO | C*T*T*A*G*G*C*C*A*A* | |
| | A | |

TABLE 3
Examples of CRISPR RNA suitable as eNA.

| CRISPR | | SEQ |
|---|---|---|
| RNA | Sequence (5′-3′) | ID |
| crR200 | GUCUUAGGCCAAAUCAUUCUCUUGAUGGUCUG | 45 |
| | UUGUGGAAGGUCCAGUUUUGAGGGGCUAUUA | |
| | CAAC | |

not underlined: ′spacer′ sequence (recognition of the target RNA)
underlined: ′direct repeat′ (binding by Cas 13b protein)

The invention further relates to the use of the method according to the invention for the identification of nucleic acids (eNAs) that direct endonucleases selected from AGO, RNase H and Cas proteins to the a-sites of target RNAs and thereby, preferably reliably, the functions of these RNA molecules, whereby the target RNAs are inactivated, for example, by endonucleolytic hydrolysis (slicing cleavage) or translation inhibition, thus resulting in silencing of the target RNA.

As mentioned above, the eNAs are preferably selected from any type of siRNAs, any type of miRNAs, any type of antisense DNA oligonucleotides, and any type of g/crRNAs. These eNAs are particularly suitable for use in, preferably reliable, RNA-mediated, DNA-mediated and/or CRISPR/Cas-mediated silencing of RNAs that may be of cellular but also pathogenic origin.

The eNAs can be used in transgenic or transient (non-transforming) form for pathogen control or for targeted, transcriptional and post-transcriptional regulation of gene expression. They are particularly suitable for use in pathogen control in plants/crops, animals/crops and humans, e.g. as virucides, bactericides, fungicides, nematicides and insecticides.

In another embodiment, the invention relates to a composition comprising at least one eNA identified by the method of the invention.

In another embodiment, the invention relates to a composition comprising at least one eNA identified by the method of the invention and optionally a carrier/excipient suitable for administration in/on plants, in nematodes, insects, oomycetes, fungi, animals and in humans.

The composition is preferably a solution that can be administered in direct form, e.g. as a nutrient solution or as an aerosol/spray. This makes it particularly easy to prevent or treat diseases in plants/crops as well as in animals and also in humans.

In another aspect, the invention provides pharmaceutical or veterinary compositions for parenteral, enteral, intramuscular, mucosal or oral administration comprising an eNA according to the invention, optionally in combination with common carriers and/or excipients. In particular, the invention relates to pharmaceutical or veterinary compositions suitable for the prevention and/or treatment of diseases in animals/domestic animals and/or in humans.

Preferably, the pharmaceutical or veterinary composition comprises at least one physiologically acceptable carrier, diluent, and/or excipient. The eNAs according to the present invention may be contained in a pharmaceutically acceptable carrier, for example, a conventional medium, such as an aqueous salt medium or buffer solution as a pharmaceutical composition for injection. Such a medium may also contain conventional pharmaceutical substances, such as pharmaceutically acceptable salts for adjusting osmotic pressure, buffers, preservatives and the like. Preferred media include physiological saline and human serum. A particularly preferred medium is PBS-buffered saline.

Other suitable pharmaceutically acceptable carriers are known to the skilled person, for example, from Remington's Practice of Pharmacy, 13th edition and J. of. Pharmaceutical Science & Technology, Vol. 52, No. 5, Sept-Oct, pp. 238-311.

In a preferred embodiment, the eNA according to the invention has one or more chemical modifications, wherein the chemical modifications may be selected from 2′-ribose modifications (2′-O-methyl, 2′-fluoro, 2′-O-(methoxyethyl) (2′-MOE)), sugar modifications (‘locked’ (LNA) or unlocked, (UNA)), ‘backbone’ modifications such as phosphorothioates (PS) or ‘peptide nucleic acids’, and sugar phosphate modifications such as morpholino/PMO (phosphorodiamidate morpholino). This increases the stability and or lifetime/half-life of the eNAs when used, e.g. in pathogen control.

The invention is explained in more detail below with reference to 5 figures and 6 examples of embodiments.

### They Show:

FIG. 1A Examples of consensus sequences of siRNAs (guide strand) determined to be optimal for plant AGO1 (left) or AGO2 (right). To determine consensus sequences, double-stranded RNAs of Tomato bushy stunt virus (TBSV) or Cucumber mosaic virus (CMV) were first processed in BYL by endogenous Dicer/DCLs to siRNAs. Total RNA was isolated from these approaches and viral siRNAs were characterized by RNA-Seq (′DCL assay′). In further approaches, either AGO1 or AGO2 protein with N-terminal FLAG-tag was produced in parallel with siRNA generation by in vitro translation of corresponding mRNAs. Subsequently, AGO/RISC loaded with viral siRNAs were isolated by immunoprecipitation using an anti-FLAG antibody, and siRNAs were purified from the complexes and characterized by RNA-Seq (‘AGO-IP’) (9). By comparing the relative abundances with which individual siRNAs were detected in each of ‘DCL-assay’ and ‘AGO-IP,’ siRNAs showing high affinity for AGO1 or AGO2 were identified. Consensus sequences for AGO1 and AGO2 were derived from the sequences of the 50 most highly enriched 21 nt siRNAs in the ‘AGO-IPs’. Positions where multiple nucleotides allow (near) maximal activity are indicated accordingly by letters placed one below the other. Similarly, this was done for AGO proteins from other organisms (e.g. fungus and human).

FIG. 1B Examples of ‘slicer assays’ performed with a 21 nt esiRNA and with consensus-optimized 21 nt esiRNAs: left with AGO1/RISC, right with AGO2/RISC. The 21 nt-long variant of gf698 siRNA specific for GFP mRNA was used as esiRNA (25, 26), and the sequences from FIG. 2A (top row) were used as consensus-optimized siRNAs. Plant AGO proteins were generated in BYL by in vitro translation of corresponding mRNAs. The translation reaction was performed in the presence of the synthetic siRNA duplexes to be tested, resulting in the incorporation of the desired siRNAs into the AGO/RISC. A radiolabeled target RNA with the appropriate matching target sequence was then added and incubated. The 21 nt long target sequences were each present in a segment of a GFP mRNA (in antisense orientation), so that the surrounding sequence was identical in all cases. Total RNA was isolated from the mounts and analyzed for cleavage products by denaturing PAGE and autoradiography. The target RNA used or the resulting cleavage products are labeled in each case.

FIG. 2 Results of ‘slicer assays’ in BYL performed with an esiRNA and AGO/RISC complexes from human, plant (Nicotiana benthamiana). and fungus ((′olletotrichum graminicola). The esiRNA used was the 21 nt long variant of the gf698 siRNA specific for GFP mRNA (25, 26). The esiRNA used contains a uridine (5′U) at the 5′ end, so all three AGO proteins were able to act with this siRNA. In addition, the esiRNA containing a 5′-adenosine was tested with the plant AGO2/RISC. The AGO proteins were generated in BYL by in vitro translation of corresponding mRNAs. The translation reaction was performed in the presence of the synthetic siRNA duplex, resulting in the incorporation of siRNA into the AGO/RISC. A radiolabeled segment of a GFP mRNA was then added as a target RNA and incubated. Total RNA was isolated from the mounts and analyzed for cleavage products by denaturing PAGE and autoradiography. The target RNA used or the resulting cleavage products are labeled in each case. A practically complete agreement of the reaction pattern can be seen—the respective cleavage products are formed in practically analogous quantities. The target RNA used and the resulting cleavage products are marked in each case.

FIGS. 3A-3D that a-sites accessible to sRNA/AGO/RISC are also accessible to ASO.
A) ‘Slicer assays’ performed in BYL with a target RNA. an esiRNA, and an antisense DNA oligonucleotide (ASO) corresponding in sequence to the guide strand of the esiRNA. Left: in the absence of translated AGO protein: Right: in the presence of translated plant AGO protein. The 21 nt long variant of gf698 siRNA specific for GFP mRNA was used as esiRNA (25. 26). An in vitro translation reaction was performed without (left) or with (right) AGO mRNA. respectively. in the presence of the indicated amounts of synthetic siRNA duplex or ASO. Subsequently. a radiolabeled segment of a GFP mRNA was added as target RNA and incubated. Total RNA was isolated from the mixtures and analyzed for cleavage products by denaturing PAGE and autoradiography. The target RNA used or the resulting cleavage products are labeled in each case. It can be seen that cleavage with ASO always occurs by RNase H activities contained in the extract. i.e. independently of AGO. whereas siRNA-mediated cleavage occurs only in the presence of additionally generated AGO. The size of the 5′-cleavage product formed in each case is identical.
B) Slicer assays in BYL performed with genomic TBSV RNA as target and different siRNAs associated to plant AGO1 (left) or AGO2 (middle). Right—analogous assay with selected ASOs having the corresponding sequence as the guide strands of corresponding siRNAs. The siRNAs used here were selected based on their high affinity for AGO1 or AGO2 as determined by ‘DCL assay’ and ‘AGO-IP’ (see FIG. 2A). AGO proteins were generated in BYL by in vitro translation of corresponding mRNAs (left. middle). The translation reaction was performed in the presence of the synthetic siRNA duplexes. resulting in the incorporation of siRNA into the AGO/RISC. ASO was used under identical conditions, but without the addition of AGO mRNA (right). Subsequently. radiolabeled genomic TBSV RNA was added as target RNA and incubated. Total RNA was isolated from the mixtures and analyzed by denaturing agarose gel and autoradiography for a decrease in the amount of target RNA and the appearance of cleavage products. The target RNA used or the cleavage products formed are labeled in each case. It can be seen that on the complex TBSV RNA. only the ASO induce cleavage, and their siRNA counterpart can also produce cleavage. The ASO are inactive whose respective siRNA counterpart is also inactive. In other words, a-sites that are accessible to SRNA/AGO/RISC are also accessible to ASO/RNase H. Sites that are not accessible to SRNA/AGO/RISC are also not accessible to ASO/RNase H.
C) Plant protection experiments with esiRNA 209 (siR209) and the corresponding ASO counterpart (ASO209) as well as controls with non-specific (GFP) siRNAs or ASO and with ASO previously found to be inefficient (see FIG. 4B). The siRNAs or ASO were rubbed onto the surface of two leaves of Nicotiana benthamiana plants together with genomic TBSV RNA (rub-in). Plants were then monitored for three weeks for the appearance of symptoms of TBSV infection. (dpi—days post infection).
D) Plant protection experiments with chemically modified and unmodified ASO 209 and control with unmodified non-specific (GFP) ASO. The experiment was performed as described in C). (MOE—2′-O-(methoxyethyl): LNA—locked nucleic acid: PTO-phosphorothioate: dpi—days post infection).

FIG. 4 the results of a ‘slicer assay’ performed with cytoplasmic HeLa S10 extract (55) with a target RNA and matching siRNA or ASO in the presence (right) and absence (left) of translated human AGO2. The 21 nt long variant of gf698 siRNA specific for GFP mRNA was used as esiRNA (25, 26). An in vitro translation reaction was performed without (left) or with (right) AGO mRNA, respectively, in the presence of synthetic siRNA duplex or ASO. Subsequently, a radiolabeled segment of a GFP mRNA was added as target RNA and incubated. Total RNA was isolated from the mixtures and analyzed for cleavage products by denaturing PAGE and autoradiography. The target RNA used or the resulting cleavage products are labeled in each case. It can be seen that analogous endonucleolytic hydrolyses also occur in HeLa extract by the respective sRNA/AGO/RISC or ASO/RNase H complexes. While cleavage with ASO occurs independently of AGO protein by RNase H activities present in the extract, siRNA activity is significantly enhanced by the amount of AGO2 increased by in vitro translation.

FIG. 5 the results of a CRISPR/Cas ‘cleavage’ experiment performed with BYL, Cas13b and TBSV RNA compared to a ‘slicer assay’ with plant AGO protein. The target sequence of CRISPR RNA 200 (crR200) used here overlaps with the target sequences of esiRNAs 186 and 209 (siR186, siR209). AGO protein as well as Cas13b from Prevotella sp. P5-125 were generated in BYL using in vitro translation of corresponding mRNAs. The translation reactions were performed in the presence of the synthetic siRNA duplexes and crRNA, respectively, resulting in the formation of the corresponding nucleic acid/endonuclease complexes. A radiolabeled region of genomic TBSV RNA was then added as a target and incubated. Total RNA was isolated from the mixtures and analyzed for cleavage products by denaturing PAGE and autoradiography. The target RNA used or the resulting cleavage products are labeled in each case. It can be seen that cleavage of viral RNA occurs in the presence of Cas13b and CRISPR RNA 200, thus the a-sites recognized by esiRNAs 186 and 209, respectively, are also targetable by crR200/Cas13b.

### Working Examples

### Example 1: Slicer Assay

The experimentally determined RNA secondary structure of the Tomato bushy stunt virus (TBSV) genome was used (30). This has segments that form functional long-range RNA-RNA interactions. This genomic viral RNA was used as a target RNA in an in vitro ‘slicer assay’ with selected 21 nt siRNAs that showed high affinity for the plant AGO proteins AGO1 or AGO2. For this purpose. the AGO proteins were generated in BYL by in vitro translation. The translation reaction was performed in the presence of the synthetic siRNA duplexes to be tested. resulting in the incorporation of the desired siRNAs into the AGO/RISC. Radioactively labeled TBSV genome was then added as a target and incubated. Total RNA was isolated from these mounts and analyzed for slicing/cleavage products by denaturing agarose gel electrophoresis and autoradiography. The position of the 5′ nucleotide of the siRNAs tested (or the 3′ end of the target sequence) was determined. The 21 nt upstream of it corresponds to the target sequence. in the middle of which endonucleolytic cleavage by RISC occurs. For siRNAs that were able to induce hydrolysis of the target RNA. corresponding cleavage fragments were detected. siRNAs that did not or barely induce hydrolysis of the target RNA could also be determined (9). On the basis of the structural analysis. it had been predicted that those regions that are largely single-stranded here are accessible to RNAi and those that are essentially double-stranded are not. No clear correspondence was found between the results of the ‘slicer assay’ and the predicted secondary structure (9).

### Example 2: Identification of Consensus Sequences of siRNAs Enhance AGO/RISC Activity

In RNA-seq analyses performed during the second step (AGO-IP) of esiRNA/ERNA screens of various target RNAs (viral RNAs and various mRNAs), a statistically relevant number of siRNAs bound by different AGO proteins were analyzed. From these analyses. guide-strand consensus sequences were generated for the respective AGOs: For each individual position of the sRNA. these reflect the nucleotide variant or variants (in the case of multiple possibilities) that was determined to make the best contribution to efficient binding of the sRNA to the corresponding AGO protein in each case (FIG. 1A). To determine the effect of the determined consensus sequences. a slicer assay was performed in which a non-optimized siRNA was tested in comparison to a consensus-optimized siRNA. The target RNA was adjusted so that both siRNAs could hybridize to it with the complete sequence in each case. In this way. the effect of the consensus sequence on AGO/RISC activity could be determined independently of the accessibility of the target RNA. It was thus shown that the slicing of the target RNA triggered by these esiRNAs/ERNAs optimized in this way can be increased again compared with the original esiRNA (FIG. 1B). The esiRNA/ERNA screen procedure has thus been improved in that it is no longer based exclusively on the empirical/experimental identification of efficiently acting esiRNAs and selection of a 5′-nucleotide adapted to the respective AGO protein. By adapting to the consensus sequence, the binding of an esiRNA/ERNA guide strand to the respective AGO protein used can be increased and thus the RNA silencing activity of the resulting sRNA/AGO/RISC can be specifically improved.

### Example 3: Demonstration that AGO/RISC Complexes from Different Organisms, with Bound esiRNAs/ERNAs of the Same Binding Sequence, Act Identically on a Target RNA

So far. it has been shown that an esiRNA/ERNA screen procedure performed in plant cytoplasmic extracts (BYL) can experimentally identify siRNAs that, as esiRNAs. can act particularly efficiently on a target RNA. e.g. the genomic RNA of a plant virus. As an important factor for the efficiency of an siRNA on a target RNA. the affinity to the AGO protein acting in each case was determined. The affinity to AGO is determined on the one hand by the 5′-nucleotide of the siRNA (29); on the other hand, as shown here according to the invention. by an optimal sequence (consensus sequence: see above). The accessibility of the target RNA to AGO/RISC has been hypothesized as another factor in the efficiency of an siRNA in RNAi. as explained. A key question that arose in this context was whether regions of a target RNA that are accessible to plant AGO/RISC are similarly so for AGO/RISC from other organisms. To test this. AGO1 and AGO2 proteins from plant (Nicotiana benthamiana). AGO2 protein from Mammalia (Homo sapiens). and AGO1 protein from Fungi ((′olletotrichum graminicola) were expressed in BYL and tested in slicer assays with a target RNA. The siRNAs used each had the identical binding sequence (the sequence that hybridizes completely with the complementary sequence of the target RNA) and a 5′ nucleotide that was matched in each case to the binding priority of the respective AGO: for plant AGO1 this was 5′U. for plant AGO2 5′A. for human AGO2 5′U or 5′A. and for fungal AGO1 5′U. Two major findings could be derived from the slicer assays performed: (i) all AGO proteins used form functional RISC in the BYL and (ii) all AGO/RISC show directly comparable slicing cleavage activity (FIG. 2). Thus. it became clear that AGO/RISC from species of three different kingdoms (Plantae. Fungi and Animalia of the Eukarya domain). when loaded with sRNAs of the same binding sequence. are active in a very comparable manner on the corresponding target RNA.

This finding is novel and unexpected. because it is shown that the accessibility of a target RNA molecule is generally given for all AGO/RISC as long as they are directed by the same nucleic acid. These can be siRNAs, but also e.g. miRNAs (28), or other types of nucleic acids (see also below).

### Example 4: Demonstrate that Regions of a Target RNA that are Accessible to SRNA/AGO/RISC are Also Accessible to ASO/RNase H

The fact that regions of a target RNA that are accessible to an sRNA/AGO/RISC are equally accessible to an sRNA/AGO/RISC from another organismal kingdom suggested that generally accessible regions, a-sites, of a complex target RNA can be detected with SRNA/AGO/RISC. This in turn led to the hypothesis that these a-sites should then be accessible to other nucleic acid/nuclease complexes, such as ASO/RNase H.

In studies conducted for this purpose, it first became clear that BYL also contain RNase H activity. Thus, it could be demonstrated for the first time that, upon supplementation of BYL with a single-stranded ASO complementary to a specific region of a target RNA, this target RNA, ASO-directed, is hydrolyzed into defined fragments. This endonucleolytic hydrolysis could thus be observed independently of the presence of a reconstituted AGO/RISC. and it generates 3′-cleavage products that differ from those of an AGO/RISC (FIG. 3A). Data obtained so far, which are not presented in detail here, indicate that both RNase H1 and RNase H2 activities are measurable in BYL (C. Gruber, unpublished data).

To test our hypothesis. ASOs were synthesized that corresponded exactly in their binding sequence to esiRNAs/ERNAs previously identified with plant AGO/RISC. In a series of experiments, it was then shown in accordance with the invention that regions previously characterized as accessible to siRNA/AGO/RISC-mediated endonucleolysis are so in an analogous manner for ASO/RNase H-mediated endonucleolysis. Thus, a viral target RNA endonucleolyzed by an esiRNA-mediated AGO/RISC is endonucleolyzed at the same site by ASO-mediated RNase H activity. In contrast, ASOs whose sequence corresponds to inactive siRNAs are not active on this target RNA (FIG. 3B). Subsequent studies in vivo demonstrated that ASO, similar to siRNAs, are able to prevent viral infections in plant. As expected, only those ASOs that correspond in sequence to esiRNAs and thus can bind to complementary a-sites in the target RNAs have an antiviral effect. In contrast, no antiviral effect was observed with ASO corresponding to siRNAs complementary to other, apparently inaccessible, regions of the target RNA used (FIG. 3C). As shown here, MOE-, LNA- and PS(PTO)-modified ASO can also be used as antiviral agents in these experiments (FIG. 3D).

### Example 5: Demonstration that sRNA/AGO/RISC and ASO/RNase H Complexes from Cytoplasmic Extracts of Plant and Human Cells, when Using sRNAs or ASO of Identical Binding Sequence, Act Identically on a Target RNA

The next question that arose was whether analogous results could be obtained in cytoplasmic extracts that were not derived from plant cells. as was BYL. To test this. it was convenient to test S10 extracts from human Hela cells under conditions similar to BYL. In HeLa S10 extracts. AGO/RISC activity can be reconstituted in vitro under very similar conditions (56): moreover, these extracts contain RNase H activity (55). In accordance with the task. slicer assays were performed in HeLa S10 with the human AGO2 protein. a target RNA. a matching esiRNA, and a derived ASO (FIG. 4). It became clear that here. analogous to the situation in BYL, the target RNA is cleaved by both endonucleolytic activities again in a very similar manner. i.e. the same 5′-cleavage product was formed in each case. In contrast to BYL. AGO/RISC activity could be reconstituted in HeLa S10 even without additionally expressed (in vitro translated) AGO; however, with additionally expressed AGO2. the activity could be increased (FIG. 4). Thus, the following became clear: In cytoplasmic extracts of completely different cell types. the folding of a complex target RNA apparently occurs in a very similar manner. The folding presumably occurs via RNA-RNA and also RNA-protein interactions with RNA-binding proteins contained in the extracts.

esiRNA/ERNA screens were performed in HeLa S10. As target RNAs, mRNAs of a human virus were used. The esiRNAs identified in vitro and (modified) ASO derived from them were subsequently tested in vivo. i.e. by application in the mouse model for a protective effect against viral infection. It was shown that mice treated with the in vitro identified esiRNAs and ASO exhibited significantly improved protection against viral infection compared to corresponding control animals.

By screening with sRNA/AGO/RISC. a-sites can be identified in a target RNA that are accessible to AGO/RISC and also to ASO/RNase H. Using BYL and other cytoplasmic extracts. e.g. human HeLa extracts. appropriately accessible regions on target RNAs can be specifically identified and these can be subjected to either sRNA- or also ASO-mediated regulation. e.g. inhibition of gene expression by endonucleolytic degradation.

Thus. cytoplasmic extracts of cells in which it is possible to reconstitute SRNA/AGO/RISC can be used according to the invention to detect the regions accessible to these complexes as well as to ASO/RNase H. a-sites. on target RNAs that are complexly folded and associated with RNA-binding proteins.

### Example 6: Demonstrate that Regions of a Target RNA that are Accessible to SRNA/AGO/RISC and ASO/RNase H are Also Accessible to CRISPR/Cas

Following the basic principle described above, it was postulated that regions of a target RNA that are accessible to sRNA/AGO/RISC are also accessible to g/crRNA-directed Cas proteins. To test this hypothesis, Cas13b protein from Prevotella sp. (P5-125) was reconstituted together with crRNAs of individual choice as endonucleolytic complexes in BYL or HeLa S10. The crRNAs were designed (FIG. 5) to overlap with regions of a target RNA previously shown to be accessible to esiRNA/AGO/RISC. In a corresponding endonucleolytic assay, it was shown that specific hydrolysis of the target RNA catalyzed by Cas13b can occur with these g/crRNAs at similar sites as with the corresponding esiRNA/AGO/RISC. That is, reconstituted sRNA/AGO/RISC and reconstituted g/crRNA/Cas 13b complexes in which the s-and g/crRNAs used hybridize to respective homologous regions on the target RNA were each used to generate degradation products (FIG. 5).

Thus, cytoplasmic extracts of cells in which it is possible to reconstitute SRNA/AGO/RISC can be used according to the invention to detect the regions accessible to these complexes as well as to ASO/RNase H, as well as to CRISPR/Cas, a-sites, on complex folded target RNAs. The target RNA can then be reliably modified in its function by RNA, DNA or CRIPR/Cas methods, e.g. inactivated by silencing.

### REFERENCE LIST

1. Shabalina S. A., Koonin E. V. (2008). Origins and evolution of eukaryotic RNA interference. Trends Ecol Evol 23, 578-587.
2. Carthew R. W., Sontheimer E. J. (2009). Origins and mechanisms of miRNAs and siRNAs. Cell 136, 642-655.
3. Guo Z., Li Y., Ding S.- W. (2019). Small RNA-based antimicrobial immunity. Nat Rev Immunol 19, 31-44.
4. Bennett C. F. (2019). Therapeutic antisense oligonucleotides are coming of age. Annu Rev Med 70, 307-21.
5. Shen X., Corey D. R. (2018). Chemistry, mechanism and clinical status of antisense oligonucleotides and duplex RNAs. Nucleic Acids Res 46, 1584-1600.
6. Moelling K., Broecker F., Russo G., Sunagawa S. (2017). RNase H as gene modifier, driver of evolution and antiviral defense. Front Microbiol 8, 1745.
7. Bevilacqua P. C., Ritchey L. E., Su Z., Assmann S. Z. (2016). Genome-Wide Analysis of RNA Secondary Structure. Ann Rev Gen 50,235-266.
8. Baltz A. G., Munschauer M., Schwanhausser B., Vasile A., Murakawa Y., Schueler M., Youngs N., Penfold-Brown D., Drew K., Milek M. et al. (2012) The mRNA-bound proteome and its global occupancy profile on protein-coding transcripts. Mol Cell 46, 674-690.
9. Gago-Zachert S., Schuck J., Weinholdt C., Knoblich M., Große I., Pantaleo V., Gursinsky T., Behrens, S.- E. (2019). Highly efficacious antiviral protection of plants by small interfering RNAs identified in vitro. Nucleic Acids Res 47, 9343-9357.
10. Vickers T. A., Crooke S.T. (2015). The rates of the major steps in the molecular mechanism of RNase H1-dependent antisense oligonucleotide-induced degradation of RNA. Nucleic Acids Res 43, 8955-8963.
11. Lucks J. B., Mortimer S. A., Trapnell C., Luo S., Aviran S., Schroth G. P., Pachter L., Doudna J. A., Arkin A. P. (2011). Multiplexed RNA structure characterization with selective 2-hydroxyl acylation analyzed by primer extension sequencing (SHAPE-Seq). Proc Natl Acad Sci USA 108, 11063-11068.
12. Rouskin S., Zubradt M., Washietl S., Kellis M., Weissman J. S. (2014). Genome-wide probing of RNA structure reveals active unfolding of mRNA structures in vivo. Nature 505, 701-705.
13. Shao Y., Chan C. Y., Maliyekkel A., Lawrence C. E., Roninson I. B., Ding Y. (2007). Effect of target secondary structure on RNAi efficiency. RNA 13, 1631-1640.
14. Tafer H., Ameres S. L., Obernosterer G., Gebeshuber C. A., Schroeder R., Martinez J., Hofacker I. L. (2008). The impact of target site accessibility on the design of effective siRNAs. Nat Biotechnol 26, 578-583.
15. Lima W. F., Vickers T. A., Nichols J., Li C., Crooke S. T. (2014). Defining the factors that contribute to on-target specificity of antisense oligonucleotides. PLOS ONE 9, e101752.
16. Ho S. P., Britton D. H., Stone B. A., Behrens D. L., Leffet L. M., Hobbs F. W., Miller J.A., TrainorG.L. (1996). Potent antisense oligonucleotides to the human multidrug resistance-1 mRNA are rationally selected by mapping RNA-accessible sites with oligonucleotide libraries. Nucleic Acids Res 24, 1901-1907.
17. Fakhr E., Zare F., Teimoori-Toolabi, L. (2016). Precise and efficient siRNA design: a key point in competent gene silencing. Cancer Gene Ther 23, 73.
18. Birmingham A. et al. (2007). A protocol for designing siRNAs with high functionality and specificity. Nat Protoc 2, 2068-2078.
19. Han Y., He F., Tan, X., Yu, H. (2017) IEEE International Conference on Bioinformatics and Biomedicine (BIBM 2017) 16-21.
20. Eastman P., Shi J., Ramsundar B., Pande V. S. (2018) Solving the RNA design problem with reinforcement learning. PLOS Comput Biol 14, e1006176.
21. Carbonell A., López C., Daròs J.- A. (2019). Fast-forward identification of highly effective artificial small RNAs against different Tomato spotted wilt virus isolates. Mol Plant Microbe Interact 32, 142-156.
22. Duan C.- G., Wang C.- H., Fang R.- X., Guo H.- S. (2008). Artificial microRNAs highly accessible to targets confer efficient virus resistance in plants. J Virol 82, 11084-11095.
23. Miozzi L., Gambino G., Burgyan J., Pantaleo V. (2013). Genome-wide identification of viral and host transcripts targeted by viral siRNAs in Vitis vinifera. Mol Plant Pathol 14, 30-43.
24. Komoda K., Naito S., Ishikawa M. (2004). Replication of plant RNA virus genomes in a cell-free extract of evacuated plant protoplasts. Proc Natl Acad Sci USA 101, 1863-1867.
25. Iki T., Yoshikawa M., Nishikiori M., Jaudal M. C., Matsumoto-Yokoyama E., Mitsuhara I., Meshi T., Ishikawa, M. (2010). In vitro assembly of plant RNA-induced silencing complexes facilitated by molecular chaperone HSP90. Mol Cell 39, 282-291.
26. Schuck J., Gursinsky T., Pantaleo V., Burgyán J., Behrens, S.- E. (2013). AGO/RISC-mediated antiviral RNA silencing in a plant in vitro system. Nucleic Acids Res 41, 5090-5103.
27. Gursinsky T., Pirovano W., Gambino G., Friedrich S., Behrens S.- E., Pantaleo V. (2015). Homeologs of the Nicotiana benthamiana antiviral ARGONAUTEl show different susceptibilities to microRNA168-mediated control. Plant Physiol 168, 938-952.
28. Pertermann R., Tamilarasan S., Gursinsky T., Gambino G., Schuck J., Weinholdt C., Lilie H., Grosse I., Golbik R. P., Pantaleo V., Behrens S.E. (2018). A viral suppressor modulates the plant immune response early in infection by regulating microRNA activity. mBio 9, e00419-18.
29. Mi S., Cai T., Hu Y., Chen Y., Hodges E., Ni F., Wu L., Li S., Zhou H., Long C., Chen S., Hannon G. J., Qi Y. (2008). Sorting of Small RNAs into Arabidopsis Argonaute Complexes Is Directed by the 5′ Terminal Nucleotide. Cell 133, 25-26
30. Wu B., Grigull J., Ore M. O., Morin S., White K. A. (2013). Global organization of a positive-strand RNA virus genome. PLOS Pathog 9, e1003363.
31. Gursinsky T., Schulz B., Behrens S.- E. (2009). Replication of Tomato bushy stunt virus RNA in a plant in vitro system. Virology 390, 250-260.
32. Carthew R. W., Sontheimer E. J. (2009). Origins and mechanisms of miRNAs and siRNAs. Cell 136, 642-655.
33. Guo Z., Li Y., Ding S.- W. (2019). Small RNA-based antimicrobial immunity. Nat Rev Immunol 19, 31-44.
34. Ding S-W (2010). RNA-based antiviral immunity. Nat Rev Immunol 10, 632-644.
35. Fukudome A., Fukuhara, T. (2017). Plant dicer-like proteins: double-stranded RNA-cleaving enzymes for small RNA biogenesis. J Plant Res 130, 33-44.
36. Parent J.- S., Bouteiller N., Elmayan T., Vaucheret, H. (2015). Respective contributions of Arabidopsis DCL2 and DCL4 to RNA silencing. Plant J 81, 223-232.
37. Meister G. (2013). Argonaute proteins: functional insights and emerging roles. Nat Rev Genet 14, 447-459.
38. Kobayashi H., Tomari Y. (2016). RISC assembly: coordination between small RNAs and Argonaute proteins. Biochim Biophys Acta 1859, 71-81.
39. Carbonell A., Carrington J.C. (2015). Antiviral roles of plant ARGONAUTES. Curr Opin Plant Biol 27, 111-117.
40. Maillard P. V., van der Veen A. G., Poirier E. Z., Reis e Sousa C. (2019) Slicing and dicing viruses: antiviral RNA interference in mammals. EMBO J 38, e100941.
41. Elbashir S. M., Harborth J., Lendeckel W., Yalcin A., Weber K., Tuschl T. (2001). Duplexes of 21-nucleotide RNAs mediate RNA interference in cultured mammalian cells. Nature 411, 494-498.
42. Setten R. L., Rossi J. J., Han S.- P. (2019). The current state and future directions of RNAi-based therapeutics. Nat Rev Drug Disc 18, 421-446.
43. Qureshi A., Tantray V. G., Kirmani A. R., Ahangar A. G. (2018). A review on currents status of antiviral siRNA. Rev Med Virol 28, e1976.
44. Dowdy S.F. (2017). Overcoming cellular barriers for RNA therapeutics. Nat Biotechnol 35, 222-229.
45. Bennett C. F. (2019). Therapeutic antisense oligonucleotides are coming of age. Annu Rev Med 70, 307-21.
46. Cerritelli S. M., Crouch R. J. (2009). Ribonuclease H: the enzymes in eukaryotes. FEBS J 276, 1494-1505.
47. Houseley J., Tollervey D. (2009). The many pathways of RNA degradation. Cell 136, 763-776.
48. Shen X., Corey D. R. (2018). Chemistry, mechanism and clinical status of antisense oligonucleotides and duplex RNAs. Nucleic Acids Res 46, 1584-1600.
49. Hagedorn P. H., Hansen B. R., Koch T., Lindow M. (2018). Managing the sequence-specificity of antisense oligonucleotides in drug discovery. Nucleic Acids Res 45, 2262-2282.
50. Nan Y., Zhang Y.- J. (2018). Antisense phosphorodiamidate morpholino oligomers as novel antiviral compounds. Front Microbiol 9, 750.
51. Wright A. V., Nuñez J. K., Doudna J. A. (2016). Biology and Applications of CRISPR Systems: Harnessing Nature's Toolbox for Genome Engineering. Cell 164, 29-44.
52. Cox D. B., Gootenberg J. S., Abudayyeh O. O., Franklin B., Kellner M. J., Joung J., Zhang F. (2017). RNA editing with CRISPR-Cas13. Science 358, 1019-1027.
53. Abudayyeh O. O., Gootenberg J. S., Konermann S., Joung J., Slaymaker I. M., Cox D. B. T., Shmakov S. et al. (2016) C2c2 is a single-component programmable RNA-guided RNA-targeting CRISPR effector. Science 353, aaf5573.
54. Sampson T. R., Saroj S. D., Llewellyn A. C., Tzeng Y.- L. Weiss, D. S. (2013) A CRISPR/Cas system mediates bacterial innate immune evasion and virulence. Nature 497, 254-257.
55. Dignam J. D., Lebovitz R. M., Roeder R. G. (1983). Accurate transcription initiation by RNA polymerase II in a soluble extract from isolated mammalian nuclei. Nucleic Acids Res 11, 1475-1489.
56. Martinez J., Patkaniowska A., Urlaub H., Lührmann R., Tuschl T. (2002). Single-stranded antisense siRNAs guide target RNA cleavage in RNAi. Cell 110, 563-574

1. Method for the detection of accessible regions (‘a-sites’) in complex RNA molecules (target RNAs), wherein nucleic acids or complexes of these nucleic acids and endonucleases associated therewith bind to the a-sites and alter the function of the target RNAs, the method comprising:
(i) provisioning of a target RNA;
(ii) performing an esiRNA/eRNA screen that identifies siRNAs that can reliably induce a functional change in this target RNA in complexes with Argonaute (AGO) proteins;
(iii) performing a subsequent assay with derived antisense DNA oligonucleotides (ASO) to determine whether they can induce a functional change in the target RNA in the presence or absence of RNase H2 and/or
(iv) performing subsequent testing with g/crRNAs derived therefrom to determine whether they can induce a functional change in the target RNA in the presence of a Cas protein; and
(v) identifying the a-sites in the target RNA, whereby nucleic acids of different types (‘eNAs’) bind to the a-sites and either alone or through an associated endonuclease alter the function of this target RNA.

2. The method according to claim 1, wherein the functional change of the target RNA is an efficient endonucleolysis or an inhibition of the translation of the target RNA.

3. The method according to claim 1, wherein the target RNA is an RNA originating from viruses, bacteria, plants, fungi, animals or humans.

4. The method according to claim 1, wherein the eNAs are selected from any kind of siRNAs (siRNAs, vsiRNAs, tasiRNAs, hpsiRNAs, natsiRNAs, vasiRNAs, hetsiRNAs, piwi RNAs, easiRNAs, phasiRNAs, endo-siRNAs) and/or any type of miRNAs and/or any type of antisense DNA oligonucleotides and/or any type of g/crRNAs.

5. The method according to claim 1, wherein the endonuclease is selected from Argonaute (AGO), RNase H and/or Cas proteins.

6. The method according to claim 1, wherein the alteration of the function of the target RNA is selected from RNA-, DNA- or CRISPR/Cas-mediated silencing.

7. The method according to claim 1, wherein the change alteration in the function of the target RNA is reliable, i.e. always occurs.

8. The method according to claim 1, wherein the eNAs have a consensus sequence that increases the affinity for the endonuclease.

9. The method according to claim 1, wherein the eNAs are any kind of siRNAs or any kind of miRNAs and the endonuclease is an AGO protein.

10. The method according to claim 1, wherein the eNAs are any type of antisense DNA oligonucleotides (ASO) and the endonuclease is an RNase H.

11. The method according to claim 1, wherein the eNAs are any type of g/crRNAs and the endonuclease is a Cas protein.

12-15. (canceled)

16. A composition comprising at least one eNA identified by the method of claim 1.

17. The composition according to claim 6, wherein the composition is a solution or an aerosol/spray.

18. The composition according to claim 16, wherein the composition is a pharmaceutical or veterinary composition.

19. The pharmaceutical or veterinary composition according to claim 18, wherein the pharmaceutical or veterinary composition is suitable for parenteral, enteral, intramuscular, mucosal or oral administration or for administration as an aerosol.

20. The composition according to claim 16, wherein the eNA comprises one or more chemical modifications; selected from 2′-ribose modifications, sugar modifications ‘backbone’ modifications, and sugar phosphate modifications.


SEQUENCE LISTING (45 sequences)

SEQ ID NO: 1 - RNA, 21 nt, Artificial Sequence
  Synthetic oligonucleotide
  misc_feature (3)..(3): n is g or a
  misc_feature (4)..(4): n is c or g
  misc_feature (5)..(5): n is c or a
  misc_feature (7)..(7): n is c or g
  misc_feature (8)..(8): n is a, g or u
  misc_feature (10)..(10): n is a or u
  misc_feature (13)..(13): n is c, g or a
  misc_feature (15)..(15): n is u, g or a
  misc_feature (18)..(18): n is u or a
  misc_feature (19)..(19): n is u or g
  misc_feature (21)..(21): n is c or a
uunnnannanaununacnnun

SEQ ID NO: 2 - RNA, 21 nt, Artificial sequence
  Synthetic Oligonucleotide
  misc_feature (4)..(4): n is g or u
  misc_feature (5)..(5): n is a, c or u
  misc_feature (7)..(7): n is g, a or c
  misc_feature (11)..(11): n is c, a or g
  misc_feature (13)..(13): n is a or u
  misc_feature (14)..(14): n is a, u or c
  misc_feature (15)..(15): n is g, c or a
  misc_feature (16)..(16): n is u or c
  misc_feature (17)..(17): n is c or u
  misc_feature (19)..(19): n is a or u
  misc_feature (20)..(20): n is u, a or c
aaannancaanannnnnunna

SEQ ID NO: 3 - RNA, 21 nt, Artificial sequence
  Synthetic oligonucleotide
uugccacaaaaucuuacuuuc

SEQ ID NO: 4 - RNA, 21 nt, Artificial sequence
  Synthetic oligonucleotide
aaagaagcaacaaagucuaua

SEQ ID NO: 5 - RNA, 21 nt, Artificial sequence
  Synthetic oligonucleotide
uaguucauccaugccaugugu

SEQ ID NO: 6 - RNA, 21 nt, Artificial sequence
  Synthetic oligonucleotide
acauggcauggaugaacuaua

SEQ ID NO: 7 - RNA, 21 nt, Artificial Sequence
  Synthetic oligonucleotide
ugauggucuccaugucgcuug

SEQ ID NO: 8 - RNA, 21 nt, Artificial Sequence
  Synthetic oligonucleotide
agcgacauggagaccaucaag

SEQ ID NO: 9 - RNA, 21 nt, Artificial Sequence
  Synthetic oligonucleotide
auucucuugauggucuccaug

SEQ ID NO: 10 - RNA, 21 nt, Artificial Sequence
  Synthetic oligonucleotide
uggagaccaucaagagaauga

SEQ ID NO: 11 - RNA, 21 nt, Artificial Sequence
  Synthetic oligonucleotide
aaaucucuuucuuaggccaaa

SEQ ID NO: 12 - RNA, 21 nt, Artificial Sequence
  Synthetic oligonucleotide
uggccuaagaaagagauuuuu

SEQ ID NO: 13 - RNA, 21 nt, Artificial Sequence
  Synthetic oligonucleotide
auaugcagacucuccacggcu

SEQ ID NO: 14 - RNA, 21 nt, Artificial Sequence
  Synthetic oligonucleotide
ccguggagagucugcauauca

SEQ ID NO: 15 - RNA, 21 nt, Artificial Sequence
  Syntheti oligonucleotide
uuucgaggcugaaucacccga

SEQ ID NO: 16 - RNA, 21 nt, Artificial Sequence
  Synthetic oligonucleotide
gggugauucagccucgaaacc

SEQ ID NO: 17 - RNA, 21 nt, Artificial Sequence
  Synthetic oligonucleotide
uuuagcccggaaaauugcacc

SEQ ID NO: 18 - RNA, 21 nt, Artificial Sequence
  Synthetic oligonucleotide
ugcaauuuuccgggcuaaaug

SEQ ID NO: 19 - RNA, 21 nt, Artificial Sequence
  Synthetic oligonucleotide
auucgccaacucaacucuauc

SEQ ID NO: 20 - RNA, 21 nt, Artificial Sequence
  Synthetic oligonucleotide
uagaguugaguuggcgaauua

SEQ ID NO: 21 - RNA, 21 nt, Artificial Sequence
  Synthetic oligonucleotide
agaugcugugacaagagcgcc

SEQ ID NO: 22 - RNA, 21 nt, Artificial Sequence
  Synthetic oligonucleotide
cgcucuugucacagcaucugg

SEQ ID NO: 23 - RNA, 21 nt, Artificial Sequence
  Synthetic oligonucleotide
aaaaacgcacugucuguaccu

SEQ ID NO: 24 - RNA, 21 nt, Artificial Sequence
  Synthetic oligonucleotide
guacagacagugcguuuuuca

SEQ ID NO: 25 - RNA, 21 nt, Artificial Sequence
  Synthetic oligonucleotide
uuagagacaguacaauuuaug

SEQ ID NO: 26 - RNA, 21 nt, Artificial Sequence
  Synthetic oligonucleotide
uaaauuguacugucucuaacc

SEQ ID NO: 27 - RNA, 21 nt, Artificial Sequence
  Synthetic oligonucleotide
auaccgguagaugugaauguc

SEQ ID NO: 28 - RNA, 21 nt, Artificial Sequence
  Synthetic oligonucleotide
cauucacaucuaccgguauca

SEQ ID NO: 29 - RNA, 21 nt, Artificial Sequence
  Synthetic oligonucleotide
uucacuguuagcuuguucccu

SEQ ID NO: 30 - RNA, 21 nt, Artificial Sequence
  Synthetic oligonucleotide
ggaacaagcuaacagugaacg

SEQ ID NO: 31 - RNA, 21 nt, Artificial Sequence
  Synthetic oligonucleotide
auucgaauucgucucaucguu

SEQ ID NO: 32 - RNA, 21 nt, Artificial Sequence
  Synthetic oligonucleotide
cgaugagacgaauucgaauca

SEQ ID NO: 33 - RNA, 21 nt, Artificial Sequence
  Synthetic oligonucleotide
aagagucugucuuacucgccu

SEQ ID NO: 34 - RNA, 21 nt, Artificial Sequence
  Synthetic oligonucleotide
gcgaguaagacagacucuuca

SEQ ID NO: 35 - DNA, 21 bp, Artificial Sequence
  Synthetic oligonucleotide
tagttcatccatgccatgtgt

SEQ ID NO: 36 - DNA, 21 bp, Artificial Sequence
  Synthetic oligonucleotide
tgatggtctccatgtcgcttg

SEQ ID NO: 37 - DNA, 21 bp, Artificial Sequence
  Synthetic oligonucleotide
aaatctctttcttaggccaaa

SEQ ID NO: 38 - DNA, 21 bp, Artificial Sequence
  Synthetic oligonucleotide
attcgccaactcaactctatc

SEQ ID NO: 39 - DNA, 21 bp, Artificial Sequence
  Synthetic oligonucleotide
aaaaacgcactgtctgtacct

SEQ ID NO: 40 - DNA, 21 bp, Artificial Sequence
  Synthetic oligonucleotide
ttagagacagtacaatttatg

SEQ ID NO: 41 - DNA, 21 bp, Artificial Sequence
  Synthetic oligonucleotide
ttcactgttagcttgttccct

SEQ ID NO: 42 - DNA, 21 bp, Artificial Sequence
  Synthetic oligonucleotide
  misc_feature (1)..(1): adenosine modified with 2′-O-methoxyethyl
  misc_feature (2)..(2): adenosine modified with 2′-O-methoxyethyl
  misc_feature (20)..(20): adenosine modified with 2′-O-methoxyethyl
  misc_feature (21)..(21): adenosine modified with 2′-O-methoxyethyl
aaatctctttcttaggccaaa

SEQ ID NO: 43 - DNA, 21 bp, Artificial Sequence
  Synthetic oligonucleotide
  misc_feature (1)..(1): Locked Nucleic Acid
  misc_feature (21)..(21): Locked Nucleic Acid
aaatctctttcttaggccaaa

SEQ ID NO: 44 - DNA, 21 bp, Artificial Sequence
  Synthetic oligonucleotide
  misc_feature (1)..(2): phosphorothioate bond
  misc_feature (2)..(3): phosphorothioate bond
  misc_feature (3)..(4): phosphorothioate bond
  misc_feature (4)..(5): phosphorothioate bond
  misc_feature (5)..(6): phosphorothioate bond
  misc_feature (6)..(7): phosphorothioate bond
  misc_feature (7)..(8): phosphorothioate bond
  misc_feature (8)..(9): phosphorothioate bond
  misc_feature (9)..(10): phosphorothioate bond
  misc_feature (10)..(11): phosphorothioate bond
  misc_feature (11)..(12): phosphorothioate bond
  misc_feature (12)..(13): phosphorothioate bond
  misc_feature (13)..(14): phosphorothioate bond
  misc_feature (14)..(15): phosphorothioate bond
  misc_feature (15)..(16): phosphorothioate bond
  misc_feature (16)..(17): phosphorothioate bond
  misc_feature (17)..(18): phosphorothioate bond
  misc_feature (18)..(19): phosphorothioate bond
  misc_feature (19)..(20): phosphorothioate bond
  misc_feature (20)..(21): phosphorothioate bond
aaatctctttcttaggccaaa

SEQ ID NO: 45 - RNA, 67 nt, Artificial Sequence
  Synthetic oligonucleotide
gucuuaggccaaaucauucucuugauggucuguuguggaagguccaguuuugaggggcuauuacaac